(19) United States
(12) Patent Application Publication
(10) Pub. No.: US 2022/0044403 A1
(43) Pub. Date: Feb. 10, 2022

(54) OPHTHALMOLOGICAL DIAGNOSIS DEVICE

(71) Applicants: CRESCO LTD., Minato-ku, Tokyo (JP); SANTEN PHARMACEUTICAL CO., LTD., Osaka-shi, Osaka (JP)

(72) Inventors: Yuji AYATSUKA, Minato-ku, Tokyo (JP); Yusuke KUDO, Minato-ku, Tokyo (JP); Takahiro IMANAKA, Osaka shi, Osaka (JP); Hideki MIYAKE, Ikoma-shi, Nara (JP)

(21) Appl. No.: 17/415,509

(22) PCT Filed: Dec. 20, 2019
(86) PCT No.: PCT/JP2019/049996

§ 371 Date: Jun. 17, 2021

(30) Foreign Application Priority Data
Dec. 21, 2018 (JP) 2018-239976

(51) Int. Cl. G06T 7/00; A61B 3/14

(52) U.S. Cl. CPC G06T 7/0014; A61B 3/145; G06T 2207/10016; G06T 2207/30041; G06T 2207/20021

(57) ABSTRACT

There is provided a technique for reducing a burden on a patient during an examination and presenting an objective examination result. An ophthalmological diagnosis device (102) for providing information to assist in making a diagnosis on a cornea includes: an input unit that receives an input of video image data from an external device (101); an output unit that outputs image data; a storage unit that stores an evaluation criterion for an injury of the cornea; and a processing unit that processes data. The processing unit generates a diagnosis image based on a position of the cornea in each still image of the video image data, the processing unit divides the cornea captured in the generated diagnosis image into a plurality of regions, and evaluates an injury in each of the divided regions based on the evaluation criterion, the processing unit generates diagnosis information (204) in which the diagnosis image and information about the evaluation on the injury in each of the regions are superimposed on each other, and the processing unit outputs the diagnosis information (204) through the output unit.

### TECHNICAL FIELD

The present disclosure relates to an ophthalmological diagnosis, more particularly, to a technique for making a diagnosis on a cornea.

### BACKGROUND ART

The surface of a pupil of an anterior eye part of an eyeball is covered with a thin film called “cornea”. The surface of the cornea is covered with a thin layer of tears called “lachrymal fluid layer”, and is protected from a dust or the like. However, in recent years, widespread use of contact lenses, long-time desk jobs with PCs (Personal Computers), and the like have led to an increased number of patients who complain symptoms of so-called dry eye involving breaking of the lachrymal fluid layer. As a dry-eye examination method, there is a method of observing a cornea for a certain period of time to make a diagnosis based on a change in state of the surface of the cornea due to drying.

Regarding the dry-eye examination method, for example, Japanese Patent Laying-Open No. 2005-211633 (PTL 1) discloses a cornea shape analysis apparatus configured as follows: “An arbitrary pattern is projected on a cornea of a subject, and reflection of the pattern from the cornea is captured in images. In the measurement, the subject is made to blink once to form a homogeneous lachrymal fluid layer on the surface of the cornea. After that, while maintaining the eyelid in an open state for about 10 seconds, a plurality of images of the pattern reflected from the cornea are recorded onto a digital memory in a time-series manner at arbitrarily determined time intervals. From the data recorded on the digital memory, an initial image just after the opening of the eyelid is employed as a reference and a cross-correlation with the images captured at the arbitral time intervals is calculated” (see [Abstract]).

Further, Japanese Patent Laying-Open No. 2004-321508 (PTL 2) discloses an ophthalmological measurement apparatus configured as follows: “The ophthalmological measurement apparatus is aligned when measurement is started. A calculation unit executes initial settings for measurement intervals, measurement times, and the like of the apparatus using a wavefront measurement unit. Triggering for the start of the measurement is provided by an input unit or the calculation unit. The calculation unit repeats the measurement of the shape of the cornea and the wavefront aberration of the cornea using the measurement unit until a measurement end time is reached. When the measurement end time is reached, a determination unit analyzes a break-up state, which is one index for determining a dry-eye state. The determination unit finds and outputs a value about the break-up, and performs an automatic diagnosis on the dry eye based on the value” (see [Abstract]).

### CITATION LIST

### Patent Literature

PTL 1: Japanese Patent Laying-Open No. 2005-211633

PTL 2: Japanese Patent Laying-Open No. 2004-321508

### SUMMARY OF INVENTION

### Technical Problem

According to each of the techniques disclosed in PTL 1 and PTL 2, the dedicated examination apparatus is required to make a diagnosis on a cornea, a great burden is imposed on the patient during the examination, and an objective and visual examination result cannot be presented. Thus, there has been required a technique for reducing a burden on a patient during an examination and presenting an objective and visual examination result.

The present disclosure has been made in view of the above background, and an object in an aspect of the present disclosure is to provide a technique for reducing a burden on a patient during an examination and presenting an objective examination result.

### Solution to Problem

An ophthalmological diagnosis device for providing information to assist in making a diagnosis on a cornea according to a certain embodiment includes: an input unit that receives an input of video image data from an external device; an output unit that outputs image data; a storage unit that stores an evaluation criterion for an injury of the cornea; and a processing unit that processes data. The processing unit generates a diagnosis image based on a position of the cornea in each still image of the video image data, the processing unit divides the cornea captured in the generated diagnosis image into a plurality of regions, and evaluates the injury in each of the divided regions based on the evaluation criterion, the processing unit generates diagnosis information in which the diagnosis image and information about the evaluation on the injury in each of the regions are superimposed on each other, and the processing unit outputs the diagnosis information through the output unit.

In a certain aspect, the generating of the diagnosis image by the ophthalmological diagnosis device include a process of extracting a reference still image from the video image data based on the position of the cornea, and combining the reference still image with another still image extracted from the video image data, by superimposing the other still image on the reference still image based on positions of an iris and the injury of the cornea captured in the reference still image.

In a certain aspect, the generating of the diagnosis image by the ophthalmological diagnosis device includes a process of comparing portions of the reference still image and the superimposed still image, the portions of the reference still image and the superimposed still image being estimated to represent the same position on the cornea, and avoiding a portion determined to have a dust from being included in the diagnosis image, the portion being determined to have the dust based on a contrast ratio or high-frequency component in each of the portions of the still images estimated to represent the same position.

In a certain aspect, the generating of the diagnosis image by the ophthalmological diagnosis device includes a process of using, for alignment of the reference still image and the superimposed still image, a wrinkle of a conjunctiva captured in each of the reference still image and the superimposed still image.

In a certain aspect, the generating of the diagnosis image by the ophthalmological diagnosis device includes a process of determining portions of the reference still image and the superimposed still image as light reflected by a conjunctiva, and avoiding the portions determined as the light reflected by the conjunctiva from being included in the diagnosis image, the portions determined as the light reflected by the conjunctiva being portions in each of which a luminance on the conjunctiva is more than a predetermined value.

In a certain aspect, the generating of the diagnosis image by the ophthalmological diagnosis device includes a process of emphasizing a portion of the captured cornea having a high contrast ratio or high-frequency component in the diagnosis image after the combining.

In a certain aspect, the generating of the diagnosis information by the ophthalmological diagnosis device includes a process of detecting position and size of the cornea captured in the diagnosis image; dividing the cornea into the plurality of regions in a form of a grid based on the detected position and size of the cornea, and evaluating the injury in each of the regions divided in the form of the grid, based on a contrast ratio or high-frequency component in the region.

In a certain aspect, the generating of the diagnosis information by the ophthalmological diagnosis device includes a process of superimposing a score on each of the regions divided in the form of the grid in the diagnosis image, the score being based on the evaluation on the injury in the region.

In a certain aspect, the generating of the diagnosis information by the ophthalmological diagnosis device includes a process of superimposing a frame line on each of the regions divided in the form of the grid in the diagnosis image, the frame line having a color that is based on the evaluation on the injury in the region.

In a certain aspect, the generating of the diagnosis information by the ophthalmological diagnosis device includes a process of calculating a comprehensive score for the evaluations on the injury in the regions.

In a certain aspect, the generating of the diagnosis information by the ophthalmological diagnosis device includes a process of blurring an iris portion captured in the diagnosis image.

In a certain aspect, the ophthalmological diagnosis device further includes a communication unit that communicates through a network. The processing unit transmits, to an external server via the communication unit, the diagnosis information to which metadata is added.

In a certain aspect, the storage unit further stores data about medicine administration. The processing unit obtains the data about the medicine administration from the storage unit, and the processing unit generates information of a medicine related to the diagnosis information, based on the diagnosis information and the data about the medicine administration.

### Advantageous Effects of Invention

According to the present disclosure, in a certain aspect, there can be provided a technique for reducing a burden on a patient during an examination and presenting an objective examination result.

The foregoing and other objects, features, aspects and advantages of the present invention will become more apparent from the following detailed description of the present invention when taken in conjunction with the accompanying drawings.

### BRIEF DESCRIPTION OF DRAWINGS

FIG. 1 is a diagram showing an exemplary configuration of an ophthalmological diagnosis system 100 according to a certain embodiment.

FIG. 2 is a diagram showing a manner of an operation of ophthalmological diagnosis system 100 according to the certain embodiment.

FIG. 3 is a diagram showing an exemplary hardware configuration of a diagnosis device 102 according to the certain embodiment.

FIG. 4 is a diagram showing an exemplary functional configuration of diagnosis device 102 according to the certain embodiment.

FIG. 5 is a diagram showing an exemplary overview of still image extraction according to the certain embodiment.

FIG. 6 is a diagram showing exemplary selection of extracted still images according to the certain embodiment.

FIG. 7 is a diagram showing exemplary selection and generation of a diagnosis still image according to the certain embodiment.

FIG. 8 is a diagram showing exemplary generation of a combined image according to the certain embodiment.

FIG. 9 is a diagram showing a first example of processing of the combined image according to the certain embodiment.

FIG. 10 is a diagram showing a second example of the processing of the combined image according to the certain embodiment.

FIG. 11 is a diagram showing a third example of the processing of the combined image according to the certain embodiment.

FIG. 12 shows exemplary processes up to the generation of the combined image by ophthalmological diagnosis system 100 according to the certain embodiment.

FIG. 13 is a diagram showing a first example of a cornea diagnosis process using a still image.

FIG. 14 is a diagram showing a second example of the cornea diagnosis process using the still image.

FIG. 15 is a diagram showing a first example of a method of editing a cornea diagnosis result using a still image.

FIG. 16 is a diagram showing a second example of the method of editing the cornea diagnosis result using the still image.

FIG. 17 is a diagram showing an exemplary diagnosis result screen.

FIG. 18 shows exemplary processes up to diagnosis and presentation of the cornea in the combined image by ophthalmological diagnosis system 100 according to the certain embodiment.

FIG. 19 is a diagram showing exemplary diagnosis data 413 according to the certain embodiment.

FIG. 20 is a diagram showing exemplary medical case data 414 according to the certain embodiment.

FIG. 21 is a diagram showing a first exemplary configuration of the ophthalmological diagnosis system according to the certain embodiment.

FIG. 22 is a diagram showing a second exemplary configuration of the ophthalmological diagnosis system according to the certain embodiment.

FIG. 23 is a diagram showing a third exemplary configuration of the ophthalmological diagnosis system according to the certain embodiment.

### DESCRIPTION OF EMBODIMENTS

Hereinafter, embodiments of technical ideas according to the present disclosure will be described with reference to figures. In the description below, the same components are denoted by the same reference characters. Their names and functions are also the same. Therefore, they will not be described repeatedly in detail.

<A. Overview of System>

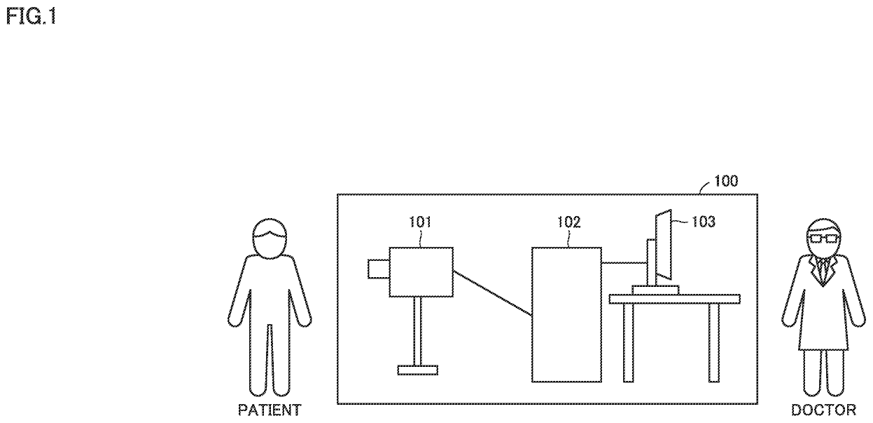

FIG. 1 is a diagram showing an exemplary configuration of an ophthalmological diagnosis system 100 according to the present embodiment. Referring to FIG. 1, an ophthalmological diagnosis system 100 according to the present embodiment includes a camera 101, a diagnosis device 102, and a monitor 103.

Camera 101 captures an image of an anterior eye part of a patient. Camera 101 is connected to diagnosis device 102 via a cable, and transmits a video image of the anterior eye part to diagnosis device 102 via the cable.

In a certain aspect, camera 101 may be connected to diagnosis device 102 via an HDMI (registered trademark) (High-Definition Multimedia Interface) cable. Alternatively, camera 101 may be connected to diagnosis device 102 via a USB (Universal Serial Bus) cable. Alternatively, camera 101 may be connected to diagnosis device 102 via an RCA cable. Alternatively, camera 101 may wirelessly communicate with diagnosis device 102 instead of a cable. Alternatively, camera 101 may temporarily store a captured video image therein and may transmit the video image to diagnosis device 102 via the Internet or the like.

In a certain aspect, camera 101 may be a camera using a CMOS (Complementary Metal-Oxide-Semiconductor) sensor or may be a camera using a CCD (Charged-Coupled Devices) sensor. Alternatively, camera 101 may be a camera of a smartphone or may be a web camera.

Diagnosis device 102 extracts still images from the video image received from camera 101. Further, diagnosis device 102 selects, from the extracted still images, a still image usable for diagnosis on a conjunctiva, or diagnosis device 102 combines a plurality of still images to generate an image by which a diagnosis on the conjunctiva can be made. Alternatively, diagnosis device 102 may encode the video image received from camera 101 into an appropriate moving image format, and then may extract a still image therefrom. Further, diagnosis device 102 quantitatively makes a diagnosis on an injury of the cornea in the still image based on pattern data of injuries of corneas.

In a certain aspect, diagnosis device 102 may be a PC, a workstation, a virtual machine on a cloud service, or dedicated hardware. Alternatively, diagnosis device 102 may be a parallel machine in which a plurality of PCs or the like are connected.

Monitor 103 is connected to diagnosis device 102 via the cable, and presents a diagnosis result of diagnosis device 102. Further, monitor 103 may present the still image of the diagnosis result as well as related diagnosis information, patient information, medicine administration proposal, and the like.

In a certain aspect, monitor 103 may be a liquid crystal monitor, an organic EL (Electro-Luminescence) display, or an OLED (Organic Light Emitting Diode) display. In a certain aspect, monitor 103 may be connected to diagnosis device 102 via an HDMI (registered trademark) cable, a D-Sub15 cable, or a DVI (Digital Visual Interface) cable.

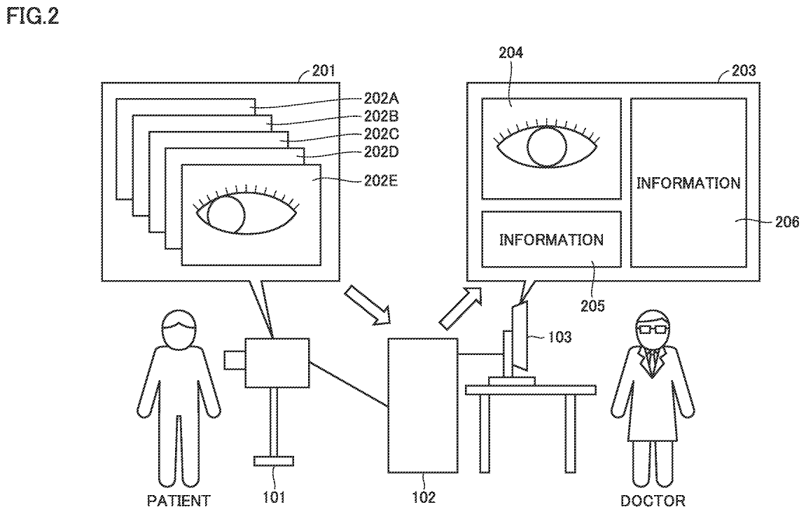

FIG. 2 is a diagram showing an manner of an operation of ophthalmological diagnosis system 100 according to the present embodiment. The following describes an overview of the operation of ophthalmological diagnosis system 100 according to the present embodiment with reference to FIG. 2. First, camera 101 captures a moving image (video image) 201 of the anterior eye part of the patient. Camera 101 transmits captured moving image 201 to diagnosis device 102.

Diagnosis device 102 encodes moving image 201 into data in a processable format, and extracts still images from moving image 201. It can be said that moving image 201 is a collection of continuous still images with each frame being regarded as a unit. As shown in FIG. 2, moving image 201 includes still images (frames) 202A to 202E.

Next, diagnosis device 102 extracts still images 202A to 202E from moving image 201. Next, from still images 202A to 202E, diagnosis device 102 selects still images to serve as candidates for use in cornea diagnosis. As an example, it is assumed that diagnosis device 102 selects still images 202A to 202C.

Next, diagnosis device 102 determines whether or not the cornea can be detected in each of selected still images 202A to 202C. When the cornea can be detected in one still image, diagnosis device 102 may directly use the still image for the cornea diagnosis. When the cornea cannot be detected in one still image, diagnosis device 102 generates a combined image for cornea detection by combining a plurality of still images. For example, when still image 202A is an image of the anterior eye part ideally captured from the front with no problem in terms of brightness, diagnosis device 102 determines that still image 202A is solely usable for the cornea diagnosis. On the other hand, when each of still images 202B and 202C is solely unusable for the cornea diagnosis, diagnosis device 102 combines still images 202B and 202C to generate a combined image for the cornea diagnosis.

Finally, diagnosis device 102 presents a cornea diagnosis still image 204, diagnosis information 205, and related information 206 on monitor 103. It should be noted that diagnosis device 102 may select one still image or a plurality of still images as cornea diagnosis still image 204.

<B. Hardware Configuration>

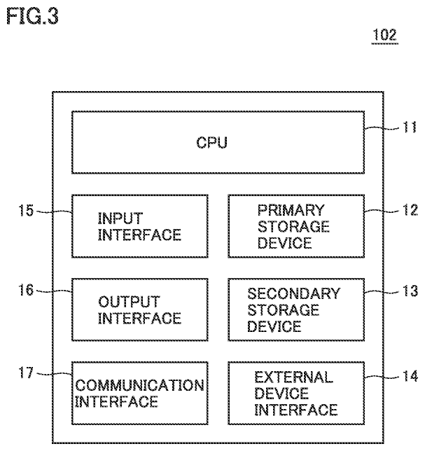

FIG. 3 is a diagram showing an exemplary hardware configuration of diagnosis device 102 according to the present embodiment. Referring to FIG. 3, diagnosis device 102 includes a CPU (Central Processing Unit) 11, a primary storage device 12, a secondary storage device 13, an external device interface 14, an input interface 15, an output interface 16, and a communication interface 17.

CPU 11 processes a program executable on diagnosis device 102, and processes data. The primary storage device stores the program executable by CPU 11 and the data to be referenced to. In a certain aspect, a DRAM (Dynamic Random Access Memory) may be used as the primary storage device.

Secondary storage device 13 stores a program, data, and the like for a long period of time. Generally, the secondary storage device is lower in rate than the primary storage device. Hence, data to be directly used by CPU 11 is placed in the primary storage device, whereas the other data is placed in the secondary storage device. In a certain aspect, a non-volatile storage device such as a HDD (hard disk drive) or a SSD (solid state drive) may be used as the secondary storage device.

External device interface 14 is used when connecting an auxiliary device to diagnosis device 102. In a certain aspect, a USB (Universal Serial Bus) interface may be used as external device interface 14. Input interface 15 is used to connect a keyboard, a mouse, or the like. In a certain aspect, a USB interface may be used as input interface 15.

Output interface 16 is used to connect an output device such as a display. In a certain aspect, HDMI (registered trademark) or DVI may be used as output interface 16. Alternatively, when diagnosis device 102 is a server machine, diagnosis device 102 may include a serial interface for communication with an external terminal.

Communication interface 17 is used to communicate with an external communication device. In a certain aspect, a LAN (Local Area Network) port, a Wi-Fi (registered trademark) (Wireless Fidelity) transmission/reception device, or the like may be used as output interface 16. Further, in a certain aspect, diagnosis device 102 may be a personal computer (PC), a workstation, or a virtual machine provided on a data center cloud.

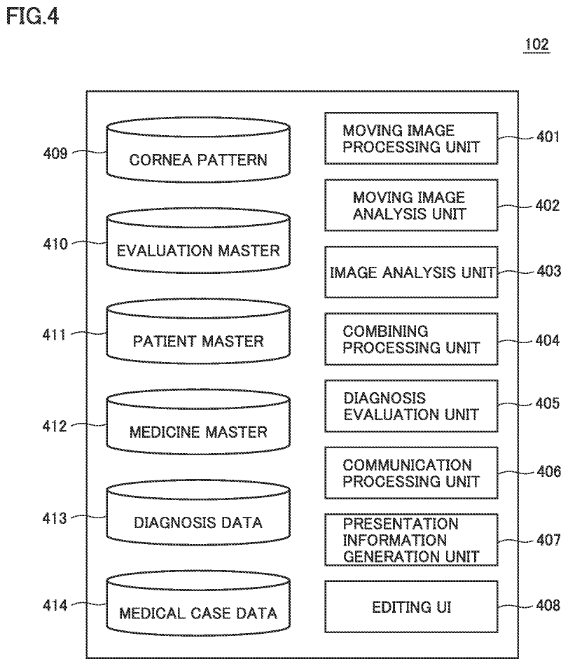

FIG. 4 is a diagram showing an exemplary functional configuration of diagnosis device 102 according to the present embodiment. Each of tables and functional units in FIG. 4 may be implemented as data and a program on the hardware of FIG. 3. Referring to FIG. 4, diagnosis device 102 includes a moving image processing unit 401, a moving image analysis unit 402, an image analysis unit 403, a combining processing unit 404, a diagnosis evaluation unit 405, a communication processing unit 406, a presentation information generation unit 407, an editing UI (User Interface) 408, a cornea pattern 409, an evaluation master 410, a patient master 411, a medicine master 412, diagnosis data 413, and medical case data 414.

Moving image processing unit 401 encodes the moving image received from camera 101. When the moving image received from camera 101 has already been encoded in a predetermined format, moving image processing unit 401 does not perform the encoding process. In the present embodiment, examples of the predetermined format includes, but not limited to, MP4, AVI, and the like.

Moving image analysis unit 402 analyzes the encoded moving image with each frame being regarded as a unit, and extracts still images from the moving image. For example, when the moving image is of 60 fps (frames per second), 60 still images are included in the moving image of one second. Moving image analysis unit 402 performs circle detection and brightness evaluation onto each still image in the moving image, and extracts only images in each of which the cornea can be detected. It should be noted that the cornea included in each still image may not be in an ideal state, and moving image analysis unit 402 extracts still images each having a certain score or higher from the moving image.

Image analysis unit 403 evaluates whether or not each of the still images extracted from the moving image is usable for the cornea diagnosis. Image analysis unit 403 makes reference to cornea pattern 409 to determine whether or not the cornea in each still image is solely usable for the cornea diagnosis. Image analysis unit 403 stores, as a diagnosis still image candidate, a still image determined to be solely usable for the cornea diagnosis. On the other hand, image analysis unit 403 sends, to combining processing unit 404, a still image determined to be solely unusable for the cornea diagnosis.

Combining processing unit 404 makes reference to cornea pattern 409 to combine the still images each solely unusable for the cornea diagnosis, thereby generating a combined image usable for the cornea diagnosis. Combining processing unit 404 sends the generated combined image to diagnosis evaluation unit 405.

Diagnosis evaluation unit 405 analyzes the still image sent from image analysis unit 403, and makes a diagnosis on the cornea using the analysis result. For example, diagnosis evaluation unit 405 makes reference to evaluation master 410 to evaluate amount and size of an injury on the surface of the cornea in the still image. Diagnosis evaluation unit 405 sends, to presentation information generation unit 407, the evaluation information and the still image used for the diagnosis.

Communication processing unit 406 performs a process for communication with an external device. Communication processing unit 406 performs updating of the data of the various types of tables as well as updating of the programs. Communication processing unit 406 may transmit the diagnosis information to an external device instead of presentation information generation unit 407 described later.

Presentation information generation unit 407 generates a screen to be presented on monitor 103. Presentation information generation unit 407 generates an image in which the evaluation information obtained from diagnosis evaluation unit 405 is superimposed on the still image used for the diagnosis. Further, presentation information generation unit 407 makes reference to patient master 411, medicine master 412, diagnosis data 413, and medical case data 414 to generate various types of information related to the diagnosis result. Patient master 411 is used to present information about the patient subjected to the diagnosis. Medicine master 412 and medical case data 414 are mainly used to present past medicine administration information and proposal information for the diagnosis result. Diagnosis data 413 is used to present a past diagnosis history.

Editing UI 408 is a user interface for a doctor or researcher to correct a diagnosis content presented on monitor 103. Diagnosis evaluation unit 405 makes reference to evaluation master 410 to automatically perform the cornea diagnosis. When the diagnosis result presented on monitor 103 is not appropriate, the doctor may operate editing UI 408 via input interface 15 to manually correct the diagnosis result. The corrected content may be fed back to evaluation master 410.

Cornea pattern 409 includes cornea pattern data. Cornea pattern 409 is used by combining processing unit 404 to recognize the cornea in the still image.

Evaluation master 410 includes pattern data for the cornea diagnosis. Evaluation master 410 is used by diagnosis evaluation unit 405 to evaluate the amount and size of the injury on the surface of the cornea captured in the still image.

Patient master 411 includes information about the patient, and is referenced to by presentation information generation unit 407 when generating the presentation screen. Medicine master 412 includes information about medicines, and is referenced to by presentation information generation unit 407 when generating the presentation screen. Diagnosis data 413 includes diagnosis information. Diagnosis data 413 is referenced to by presentation information generation unit 407 when generating the presentation screen. Medical case data 414 includes past medical case information. Medical case data 414 is referenced to by presentation information generation unit 407 when presenting the information related to the diagnosis result.

<C. Procedure in Generation of Diagnosis Image>

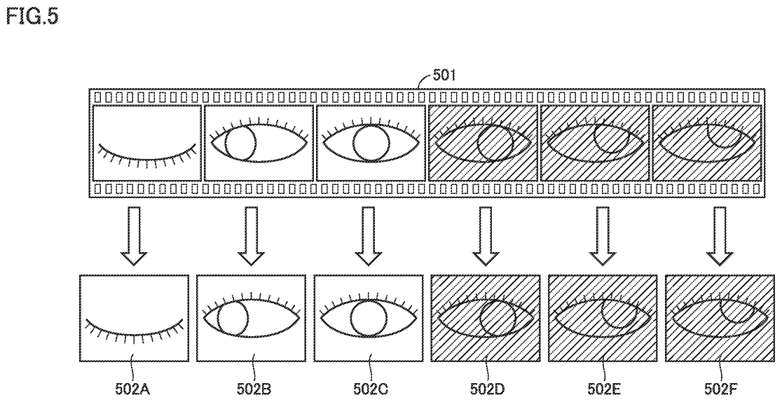

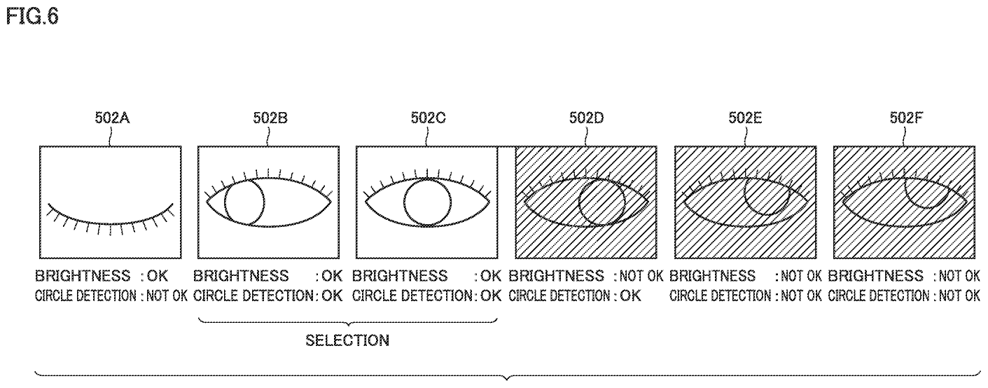

FIG. 5 is a diagram showing an exemplary overview of still image extraction according to the present embodiment. The following describes a flow of the still image extraction by moving image analysis unit 402 with reference to FIG. 5. Moving image 501 includes six frames. Moving image analysis unit 402 divides moving image 501 into the frames, i.e., still images 502A to 502F. Moving image analysis unit 402 performs a below-described process shown in FIG. 6 onto divided still images 502A to 502F. It should be noted that when the size of the moving image is large, moving image analysis unit 402 may extract a still image of each portion of the moving image. Since moving image analysis unit 402 divides the moving image, which has a large amount of information and requires a long time to analyze, into the individual still images, the processes of the other functional units are facilitated and are speeded up.

FIG. 6 is a diagram showing exemplary selection of extracted still images according to the present embodiment. Moving image analysis unit 402 determines whether or not each still image has a portion in which a circle having a certain size or larger can be detected, or determines whether or not the still image has a certain brightness or higher. In this way, moving image analysis unit 402 deletes a still image apparently having no conjunctiva captured therein and a too dark still image. In the example shown in FIG. 6, moving image analysis unit 402 selects still images 502B, 502C as the diagnosis still image candidates and deletes the other images. In each of still images 502B, 502C, the brightness and the circle detection result have values of more than or equal to predetermined values.

Moving image analysis unit 402 generates the still images and excludes the unnecessary still images from the large amount of still images by simply making determination in accordance with the brightness, the circle detection, and the like. In this way, moving image analysis unit 402 reduces used areas of primary storage device 12 and secondary storage device 13, and reduces workloads of the other functional units.

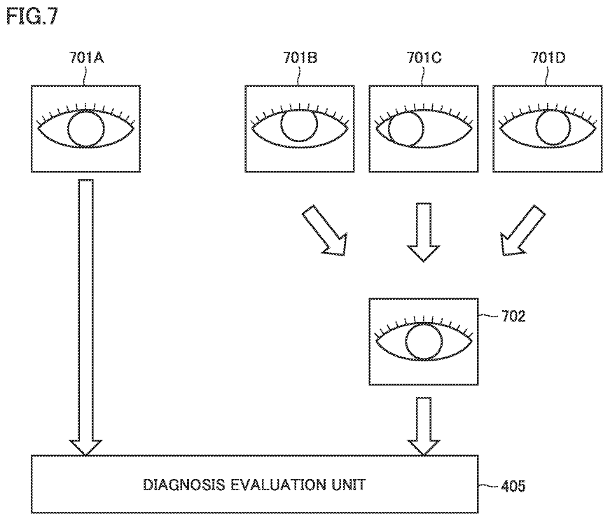

FIG. 7 is a diagram showing exemplary selection and generation of the diagnosis still image according to the present embodiment. The following describes overviews of a method of selecting the diagnosis still image and a method of generating a diagnosis still image with reference to FIG. 7. It is assumed that image analysis unit 403 obtains still images 701A to 701D from moving image analysis unit 402.

When image analysis unit 403 obtains still images 701A to 701D, image analysis unit 403 makes reference to cornea pattern 409 to determine whether or not the cornea in each of still images 701A to 701D is solely usable for the cornea diagnosis. In the example shown in FIG. 7, image analysis unit 403 determines that still image 701A is solely usable for the cornea diagnosis, and sends still image 701A to diagnosis evaluation unit 405. On the other hand, image analysis unit 403 determines that still images 701B to 701D are not each usable solely for cornea diagnosis, and sends still image 701A to combining processing unit 404.

Combining processing unit 404 obtains the plurality of still images each unusable solely for the cornea diagnosis, and generates a combined image solely usable for the cornea diagnosis. In the example shown in FIG. 7, combining processing unit 404 combines still images 701B to 701D obtained from image analysis unit 403, so as to generate a combined image 702 solely usable for the cornea diagnosis. It should be noted that details of the combining method will be described later.

Image analysis unit 403 selects, through pattern matching or the like, the still image solely usable for the cornea diagnosis, thereby facilitating the below-described cornea diagnosis. Further, since combining processing unit 404 produces, based on the plurality of still images, the combined image solely usable for the cornea diagnosis, an effective cornea diagnosis can be facilitated even with still images obtained by an ordinary camera.

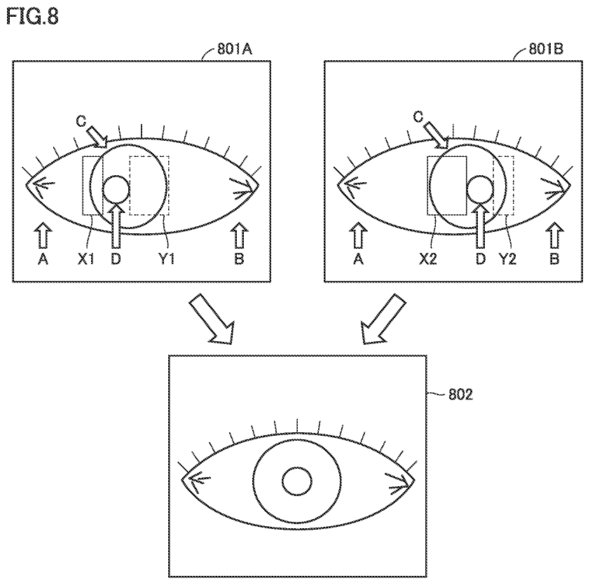

FIG. 8 is a diagram showing exemplary generation of the combined image according to the present embodiment. The following describes a procedure in the generation of the combined image by combining processing unit 404 with reference to

FIG. 8. It is assumed that combining processing unit 404 obtains still images 801A and 801B from image analysis unit 403. In each of still images 801A and 801B, a conjunctiva A, a conjunctiva B, a cornea C, and a pupil D are captured.

In still image 801A, cornea C is inclined to the left side when viewed from the front, and the entirety of a region X1 is not captured. Hence, still image 801A is not unusable solely for the cornea diagnosis. On the other hand, in still image 801B, cornea C is inclined to the right side when viewed from the front, and the entirety of a region Y2 is not captured. Also, still image 801B is not usable solely for the cornea diagnosis.

Combining processing unit 404 aligns the respective anterior eye parts in the still images based on the shapes and relative positions of conjunctiva A, conjunctiva B, cornea C, and pupil D. In the example shown in FIG. 8, combining processing unit 404 determines that region X1 of still image 801A and region X2 of still image 801B are the same region. Further, combining processing unit 404 determines that region Y1 of still image 801A and region Y2 of still image 801B are the same region.

Combining processing unit 404 collects and combines clearly captured portions from still images 801A and 801B, so as to generate a combined image 802. For example, combining processing unit 404 selects region Y1 from still image 801A and selects region X2 from still image 801B and combines them.

It should be noted that when combining a plurality of still images, combining processing unit 404 may employ one still image as a reference still image and may superimpose another still image on the reference still image based on a combination of the positions of the cornea, the iris, the injury of the cornea, and the conjunctiva in the reference still image. When it is difficult to superimpose the still images only using the cornea, combining processing unit 404 may use a wrinkle of the conjunctiva at the end portion of the anterior eye part for the sake of alignment.

Further, combining processing unit 404 may estimate and correct the inclination of the combined region. It is assumed that region Y1 and region X2 are used in combined image 802. In this case, based on the shapes and relative positions of conjunctiva A, conjunctiva B, cornea C, and pupil D, combining processing unit 404 may estimate at what degrees of angles region Y1 and region X2 are inclined to the left and right sides with respect to the front, and may combine region Y1 and region X2 after distorting them so as to attain a shape close to the shape when oriented to the front.

Based on the shapes and relative positions of the regions specific to the anterior eye part of the human being, combining processing unit 404 generates, from the plurality of still images, the combined image of the anterior eye part oriented to the front. In this way, combining processing unit 404 can appropriately generate the combined image even when the patient's face is moved during the image capturing, thereby facilitating the cornea diagnosis process.

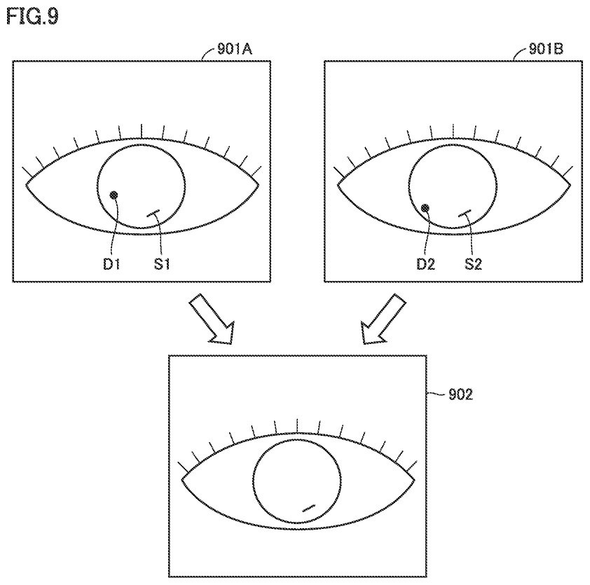

FIG. 9 is a diagram showing a first example of processing of the combined image according to the present embodiment. The following describes a first procedure in the processing of the combined image by combining processing unit 404 with reference to FIG. 9. In a certain aspect, it is assumed that combining processing unit 404 obtains still images 901A and 901B from image analysis unit 403. In still image 901A, foreign objects D1 and S1 are captured to be on the surface of the cornea. In still image 901B, foreign objects D2 and S2 are captured to be on the surface of the cornea. The position of the cornea in still image 901A and the position of the cornea in still image 901B are substantially the same.

First, combining processing unit 404 makes reference to cornea pattern 409 to specify foreign objects D1, D2, S1, and S2 on the cornea in still images 901A and 901B through image recognition.

Next, based on the relative positions of the conjunctiva, cornea, pupil, and the like in still images 901A and 901B, combining processing unit 404 estimates that foreign object D1 in still image 901A and foreign object D2 in still image 901B are the same foreign object. Similarly, based on the relative positions of the conjunctiva, cornea, pupil, and the like in still images 901A and 901B, combining processing unit 404 estimates that foreign object S1 of still image 901A and foreign object S2 of still image 901B are the same foreign object.

In the example shown in FIG. 9, it is understood that the relative positions of foreign object D1 and foreign object D2 when viewed from the cornea and the conjunctiva are deviated from each other, whereas the relative positions of foreign object S1 and foreign object S2 when viewed from the cornea and the conjunctiva are not substantially deviated from each other. In view of this, combining processing unit 404 estimates that each of foreign objects D1, D2 is a “dust” and each of foreign objects S1, S2 is an “injury”. Then, when generating combined image 902 from still images 901A, 901B, combining processing unit 404 deletes foreign objects D1, D2 each representing the “dust” while foreign objects S1, S2 each representing the “injury” are combined to remain. Combining processing unit 404 determines the deviation of the positions of the foreign objects based on a difference in contrast ratio or high-frequency component between the corresponding portions of the compared still images. By performing the process of FIG. 9 simultaneously with the process of FIG. 8, combining processing unit 404 removes the unnecessary dust from the cornea of the combined image when making the cornea diagnosis, thereby facilitating the cornea diagnosis.

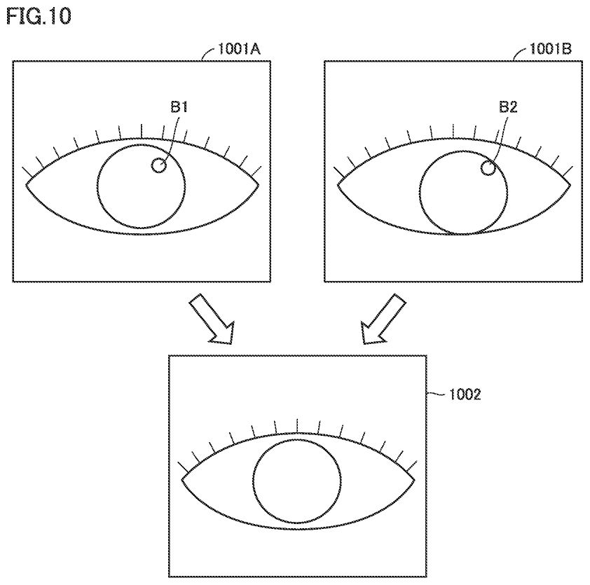

FIG. 10 is a diagram showing a second example of the processing of the combined image according to the present embodiment. The following describes a second procedure in the processing of the combined image by combining processing unit 404 with reference to FIG. 10. It is assumed that combining processing unit 404 obtains still images 1001A and 1001B from image analysis unit 403. In still image 1001A, a light reflection B1 is captured. In still image 1001B, a light reflection B2 is captured.

Combining processing unit 404 determines that a portion (light reflection B1) of still image 1001A having a luminance of more than a predetermined luminance is light reflection. Similarly, combining processing unit 404 determines that a portion (light reflection B2) of still image 1001B having a luminance of more than a predetermined luminance is light reflection.

Since the position of the cornea in still image 1001A is different from the position of the cornea in still image 1001B, the position of the light reflection on the cornea in still image 1001A is also different from the position of the light reflection on the cornea in still image 1001B (the position of light reflection B1 and the position of light reflection B2 are different). Combining processing unit 404 combines still images 1001A and 1001B in which light is reflected at different positions on the cornea, and generates a combined image 1002 in which the respective light reflections are removed. By performing the process of FIG. 10 simultaneously with the process of FIG. 8, combining processing unit 404 removes the unnecessary light reflection from the cornea of the combined image when making the cornea diagnosis, thereby facilitating the cornea diagnosis.

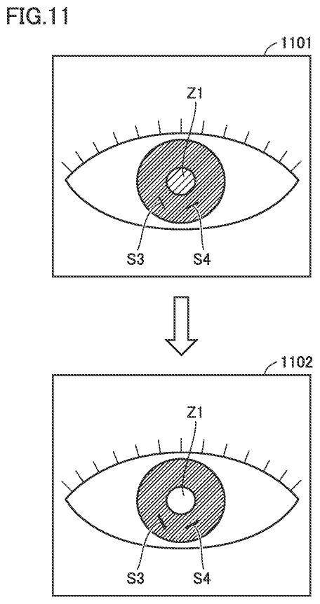

FIG. 11 is a diagram showing a third example of the processing of the combined image according to the present embodiment. The following describes a third procedure in the processing of the combined image by combining processing unit 404 with reference to FIG. 11. In a certain aspect, it is assumed that combining processing unit 404 obtains still image 1101 from image analysis unit 403. In still image 1101, injuries S3, S4 on the cornea, and a pupil Z1 are captured.

Combining processing unit 404 performs an emphasizing process onto a portion of the still image involving large changes in lightness/darkness, contrast ratio, and sharpness. In the example shown in FIG. 11, combining processing unit 404 mainly emphasizes injuries S3, S4 on the cornea, and pupil Z1. It should be noted that since the iris serves as an information source for identifying an individual person, it may be desirable to blur the iris in view of privacy protection. When the iris information is not necessary for the cornea diagnosis, combining processing unit 404 may perform a blurring process onto the iris. Further, the emphasizing process on the still image and the blurring of the iris are not essential in the cornea diagnosis. Therefore, the user may be able to employ a setting of diagnosis device 102 to make switching as to whether to perform the emphasizing process or the iris blurring process.

Combining processing unit 404 may perform the process of FIG. 11 simultaneously with the process of FIG. 8. Generally, the sharpness of an image is high in the vicinity of a boundary between a portion with an injury and a portion with no injury on the cornea. Combining processing unit 404 performs the emphasizing process onto the portion of the still image involving the large change in lightness/darkness or sharpness so as to render the injury noticeable, thereby facilitating the cornea diagnosis. Further, combining processing unit 404 performs the blurring process onto a portion providing personal information such as the iris, thereby facilitating privacy protection.

<D. Flow of Procedure in Generation of Diagnosis Image>

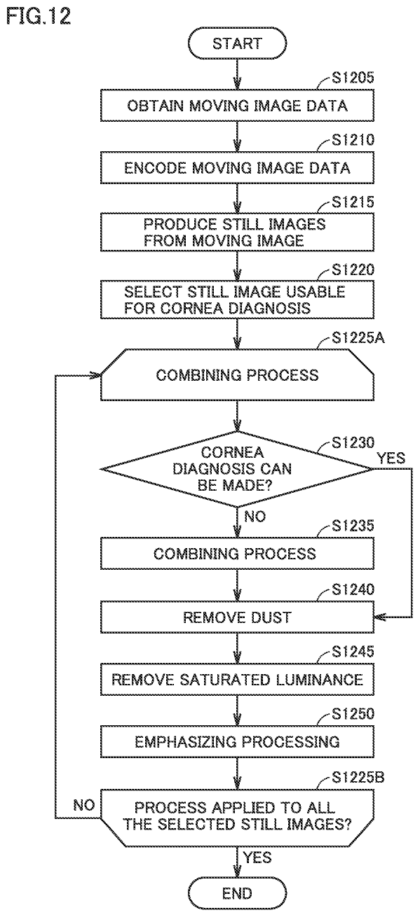

FIG. 12 shows exemplary processes up to the generation of the combined image by ophthalmological diagnosis system 100 according to the present embodiment. The following describes a procedure in the execution of the process of generating the combined image on the hardware of diagnosis device 102 with reference to FIG. 12. In a certain aspect, the various types of tables of FIG. 4 may be stored as data in secondary storage device 13 and may be referenced to by CPU 11. In a certain aspect, the various types of functions illustrated in FIG. 4 may be implemented by loading a program on primary storage device 12 and executing the program by CPU 11.

In a step S1205, CPU 11 serves as moving image processing unit 401 to obtain a moving image from camera 101. It should be noted that CPU 11 may perform processes of a step S1210 and subsequent steps during the obtainment of the moving image from camera 101. Further, CPU 11 may divide, for each certain reproduction time, the data obtained from camera 101, may temporarily store the divided data into primary storage device 12 or secondary storage device 13, and may apply the processes of step S1210 and subsequent steps onto each divided moving image. Further, after the process of step S1205, CPU 11 may delete the moving image temporarily stored in primary storage device 12 or secondary storage device 13.

In step S1210, CPU 11 serves as moving image processing unit 401 to encode the moving image obtained from camera 101 into a predetermined format and temporarily store the moving image into primary storage device 12 or secondary storage device 13. It should be noted that after the process of step S1210, CPU 11 may delete the moving image encoded in the predetermined format and temporarily stored in primary storage device 12 or secondary storage device 13. Further, when the moving image is encoded in the predetermined format by camera 101 in advance, CPU 11 may skip the process of step S1210.

In a step S1215, CPU 11 serves as moving image analysis unit 402 to generate still images from the moving image encoded in the predetermined format with each frame being regarded as a unit. Further, CPU 11 determines whether or not each of the still images has a portion in which a circle having a certain size or larger can be detected, and determines whether or not the brightness of each still image is more than or equal to a certain brightness, etc. Further, CPU 11 deletes a still image apparently having no conjunctiva captured therein and a too dark still image, and temporarily stores, into primary storage device 12 or secondary storage device 13, only a still image usable for the cornea diagnosis. The process of step S1215 corresponds to each of the processes in FIGS. 5 and 6.

In a step S1220, CPU 11 serves as image analysis unit 403 to select, from the still images generated in step S1215, a still image usable for the cornea diagnosis. Further, from the still images generated in step S1215, CPU 11 selects a still image to be used to generate a combined image for the cornea diagnosis. When selecting a still image, CPU 11 makes reference to cornea pattern 409 from secondary storage device 13 for the sake of use in pattern matching or the like for the selection of the still image.

CPU 11 serves as combining processing unit 404 to perform a loop process from steps S1225A to S1225B. In the loop from steps S1225A to S1225B, CPU 11 performs the below-described combining process onto M still images selected in step S1220. In the description below, the process onto the N-th still image will be described as an example.

In a step S1230, CPU 11 serves as combining processing unit 404 to determine whether or not the cornea diagnosis can be made by using the N-th still image solely. When making the determination, CPU 11 makes reference to cornea pattern 409 from secondary storage device 13. When CPU 11 determines that the cornea diagnosis can be made by using the N-th still image solely (YES in step S1230), CPU 11 transitions the control to a step S1235. When CPU 11 determines that the cornea diagnosis cannot be made by using the n-th still image solely (NO in step S1230), CPU 11 transitions the control to a step S1240.

It should be noted that a determination criteria in step S1230 is stricter than a determination criteria in step S1220. For example, in step S1220, CPU 11 selects a still image usable to generate a combined image, even when the cornea is not completely captured therein, whereas in step S1230, CPU 11 selects only a still image solely usable for the cornea diagnosis.

In step S1235, CPU 11 serves as combining processing unit 404 to performs a combining process to combine the N-th still image with other still image(s). For example, when the cornea diagnosis cannot be made by using the N-1-th still image solely and by using the N-2-th still image solely, CPU 11 combines the N-2-th to N-th still images to generate a combined image usable for the cornea diagnosis. It should be noted that any number of still images may be combined. The processes of steps S1220 to S1235 correspond to the processes in FIGS. 7 and 8.

In step S1240, CPU 11 serves as combining processing unit 404 to perform dust removal processing onto the N-th still image. The process of step S1240 corresponds to the process of FIG. 9.

In step S1245, CPU 11 serves as combining processing unit 404 to remove, from the N-th still image, a portion having a saturated luminance The process of step S1245 corresponds to the process of FIG. 10. It should be noted that in the processes of steps 51235 to 51245, in order to remove the dust or saturated luminance, CPU 11 may also perform the combining process to combine, with another still image, the still image determined to be solely usable for the cornea diagnosis in step S1220.

In step S1250, CPU 11 serves as combining processing unit 404 to perform emphasizing processing onto the N-th still image with regard to an injury or the like on the cornea. It should be noted that the process of step S1250 is not essential in the cornea diagnosis. Therefore, CPU 11 may perform the process of step S1250 only when an emphasizing processing instruction is received from the user via input interface 15. The process of step S1250 corresponds to the process of FIG. 11.

In step S1225B, CPU 11 serves as combining processing unit 404 to determine whether or not all the still images selected in step S1220 have been subjected to the processes in the loop of steps S1225A to S1225B. When CPU 11 determines that the processes in the loop of steps S1225A to S1225B have not been completed for all the still images selected in step S1220 (NO in step S1225B), CPU 11 transitions the control to step S1225A. Then, CPU 11 performs the processes in the loop of steps S1225A to S1225B onto the N+1-th still image. When CPU 11 determines that the processes in the loop of steps S1225A to S1225B have been completed for all the still images selected in step S1220 (YES in step S1225B), CPU 11 ends the process.

By performing the processes of FIG. 12, CPU 11 can appropriately extract still images usable for the cornea diagnosis from a moving image, which is unsuitable for analysis due to a large amount of information. Further, CPU 11 combines and processes the extracted still images to facilitate the below-described cornea diagnosis process. Further, with the still image combining process and the still image processing process of FIG. 12, the cornea diagnosis can be made even with a moving image obtained through image capturing with no special device. This leads to a reduced physical burden on the patient subjected to the examination.

<E. Procedure in Generation of Diagnosis Result>

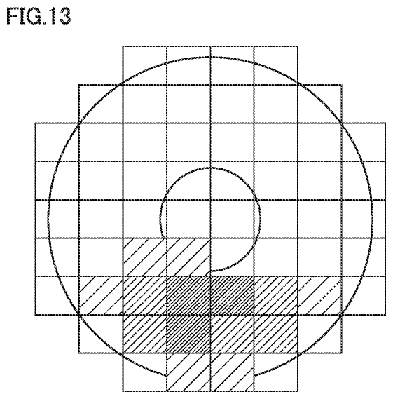

FIG. 13 is a diagram showing a first example of the cornea diagnosis process using a still image. The following describes a diagnosis using the combined image for the cornea diagnosis with reference to FIG. 13. The combined image for the cornea diagnosis is generated by the processes of FIGS. 5 to 11.

Diagnosis evaluation unit 405 divides, into certain regions, the cornea captured in the combined image for the cornea diagnosis generated by the processes of FIGS. 5 to 11. In the example shown in FIG. 13, diagnosis evaluation unit 405 divides the cornea into regions in the form of a grid of squares. It should be noted that the diagram shown in FIG. 13 is exemplary, and the cornea may not be divided into such square regions.

Diagnosis evaluation unit 405 makes reference to evaluation master 410 to evaluate the size of an injury in each region. Evaluation master 410 includes a parameter for pattern matching and is used to evaluate the size of the injury on the cornea. In accordance with the amount of injury, diagnosis evaluation unit 405 provides different colors to the squares of the grid to facilitate a visual determination on a distribution of the injury. Alternatively, diagnosis evaluation unit 405 may provide translucent colors to the squares of the grid such that the injury in the combined image can be also seen. Alternatively, diagnosis evaluation unit 405 may provide colors only to the frames of the squares of the grid. Further, the combined image, the squares of the grid, and the colors of the squares of the grid may be individually stored in primary storage device 12 or secondary storage device 13 as layer information.

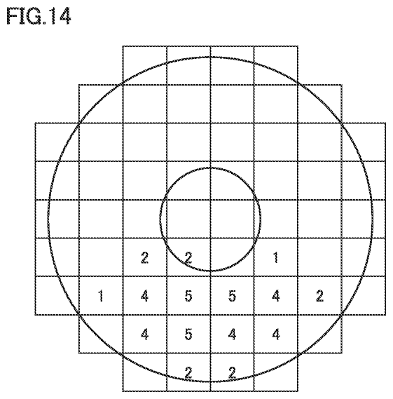

FIG. 14 is a diagram showing a second example of the cornea diagnosis process using the still image. The following describes a diagnosis using the combined image for the cornea diagnosis with reference to FIG. 14. The combined image for the cornea diagnosis is generated by the processes of FIGS. 5 to 11.

Diagnosis evaluation unit 405 divides, into certain regions, the cornea captured in the combined image for the cornea diagnosis generated by the processes of FIGS. 5 to 11. Diagnosis evaluation unit 405 evaluates the injury in each of the squares of the grid in the same manner as in the process of FIG. 13. Diagnosis evaluation unit 405 presents an evaluation value for each of the squares of the grid based on the evaluation on the injury. It should be noted that the diagram shown in FIG. 14 is exemplary, and the evaluation value is not limited to an integer and may be expressed by a decimal value, a percentage, or a symbol such as an alphabet. Further, for each of the squares of the grid, diagnosis evaluation unit 405 may combine the provision of different colors in FIG. 13 with the presentation of evaluation values in FIG. 14 based on the evaluation on the injury. Further, diagnosis evaluation unit 405 may perform the diagnosis for each region shown in FIG. 13 or 14 and calculate a comprehensive evaluation for the respective evaluations on the injury in the regions.

An injury of the cornea is a cause of dry eye. Therefore, in a diagnosis on dry eye, it is very important to quantitatively evaluate the injury of the cornea. Diagnosis evaluation unit 405 performs the diagnosis process of FIG. 13 or 14 onto the still image so as to perform a “quantitative evaluation” on the injury in each of the certain regions of the cornea, and can generate a “visual evaluation result”.

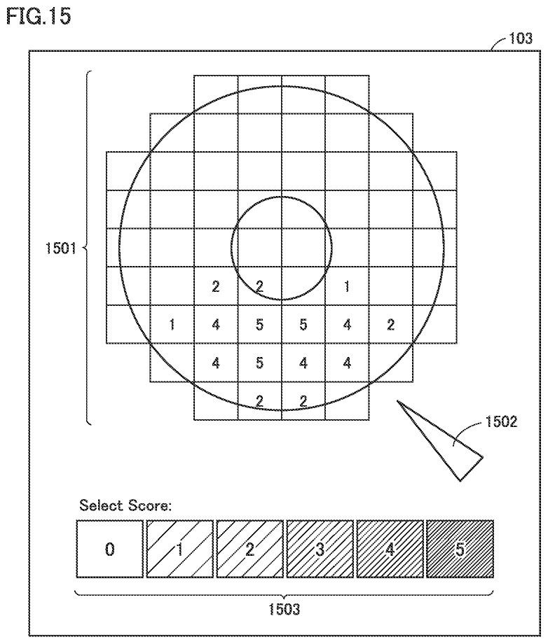

FIG. 15 is a diagram showing a first example of a method of editing the cornea diagnosis result using the still image. The following describes a procedure in editing the diagnosis result by operating editing UI 408 via input interface 15 with reference to FIG. 15.

Editing UI 408 includes a cornea diagnosis result 1501, a cursor 1502, and an evaluation value selector 1503. The user may operate cursor 1502 via input interface 15, may select an evaluation value from evaluation value selector 1503, and may rewrite an evaluation value by selecting a corresponding square of the grid in cornea diagnosis result 1501.

It should be noted that editing UI 408 in FIG. 15 is exemplary, and the editing of the diagnosis result is not limited to this method. Instead of the cursor, a touch panel or the like may be used as an input device, or an evaluation value may be entered using a voice input or the like.

Editing UI 408 provides a function of editing the evaluation value of the diagnosis result as shown in FIG. 15. In this way, the user can flexibly correct an incorrect evaluation made on the injury of the cornea by diagnosis device 102. Further, CPU 11 feeds back the correction of the diagnosis result to evaluation master 410 so as to further improve precision of the diagnosis on the cornea.

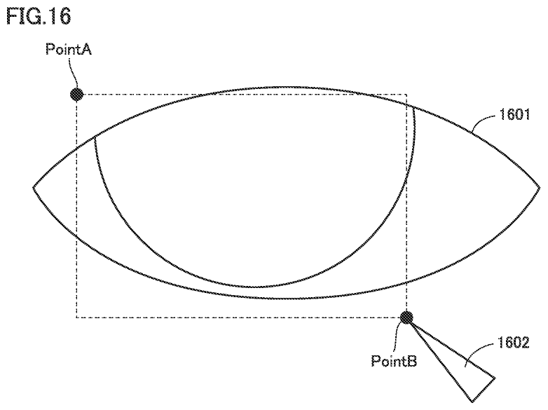

FIG. 16 is a diagram showing a second example of the method of editing the cornea diagnosis result using the still image. The following describes a procedure in editing the diagnosis result by operating editing UI 408 via input interface 15 with reference to FIG. 16.

Editing UI 408 includes an anterior eye part image 1601 and a cursor 1602. The user may select a region targeted for the diagnosis by operating cursor 1602 via input interface 15 to select arbitrary two points, i.e., Point A and Point B.

It should be noted that editing UI 408 in FIG. 16 is exemplary, and the editing of the diagnosis result is not limited to this method. Instead of the cursor, a touch panel or the like may be used, or a stylus may be used to fill a range intended to be selected.

Editing UI 408 provides a function of editing the diagnosis range of the cornea as shown in FIG. 16. In this way, the user can flexibly make a correction when diagnosis device 102 cannot appropriately recognize the range to be diagnosed. Further, CPU 11 feeds back the correction of the diagnosis result to cornea pattern 409 to further improve precision in the selection of the range on the cornea.

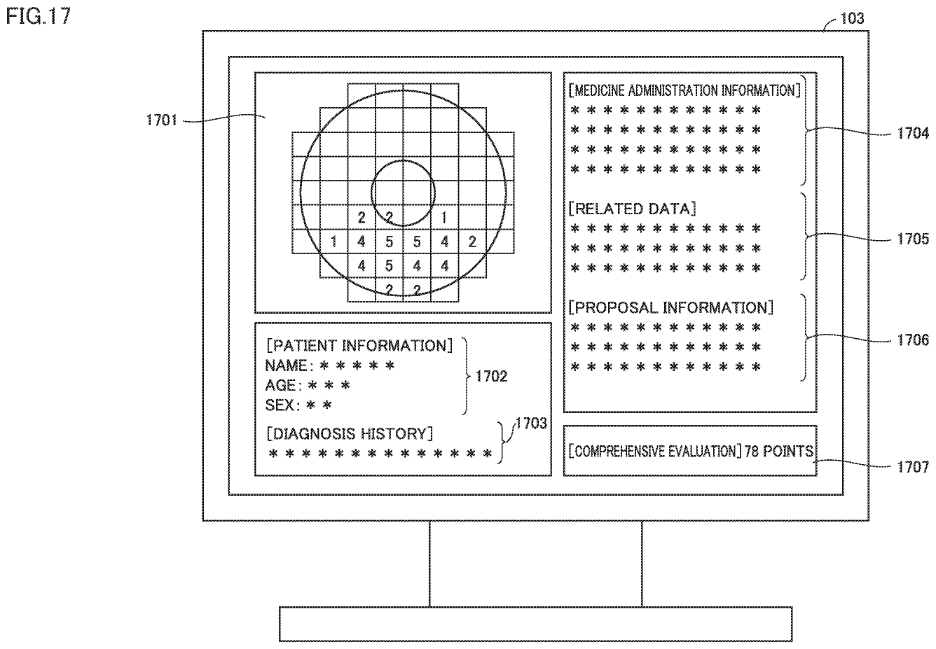

FIG. 17 is a diagram showing an exemplary diagnosis result screen. The following describes the diagnosis result screen presented on monitor 103 with reference to FIG. 17. Referring to FIG. 17, the diagnosis result screen includes a cornea diagnosis image 1701, patient information 1702, a diagnosis history 1703, medicine administration information 1704, related data 1705, proposal information 1706, and a comprehensive evaluation 1707. Presentation information generation unit 407 presents the screen shown in FIG. 17 on monitor 103 based on the cornea diagnosis result.

Cornea diagnosis image 1701 is a presented image in which the combined image for the cornea diagnosis generated by the processes of FIGS. 5 to 11 and the region information and evaluation values generated by the processes of FIGS. 13 to 16 are superimposed on each other.

Patient information 1702 is information about the patient subjected to the cornea diagnosis. Presentation information generation unit 407 makes reference to patient master 411 to obtain patient information 1702. Diagnosis history 1703 is information about past diagnosis for the patient subjected to the cornea diagnosis. Presentation information generation unit 407 makes reference to diagnosis data 413 to obtain diagnosis history 1703.

Medicine administration information 1704 is information about a medicine administered to the patient subjected to the cornea diagnosis. Presentation information generation unit 407 makes reference to medicine master 412 to obtain medicine administration information 1704. Related data 1705 is information about past related medical cases and medicine administrations. Proposal information 1706 is proposal information or the like about medicine administration content and treatment method considered to be effective in view of past medical cases. Presentation information generation unit 407 makes reference to medicine master 412 and medical case data 414 to generate related data 1705 and proposal information 1706. Comprehensive evaluation 1707 is a comprehensive evaluation that is based on the diagnosis result for each region in FIG. 13 or 14 and serves as an index for measuring a degree of severity of the injury of the cornea. It should be noted that the diagnosis result screen of FIG. 17 is exemplary and the presentation content of the diagnosis result screen is not limited to this example.

As shown in FIG. 17, presentation information generation unit 407 presents the useful information such as the medicine administration information and the past medical case information together with the visual cornea diagnosis result. In this way, diagnosis device 102 can more appropriately support decision making by a doctor or researcher.

<F. Flow of Procedure in Generation of Diagnosis Result>

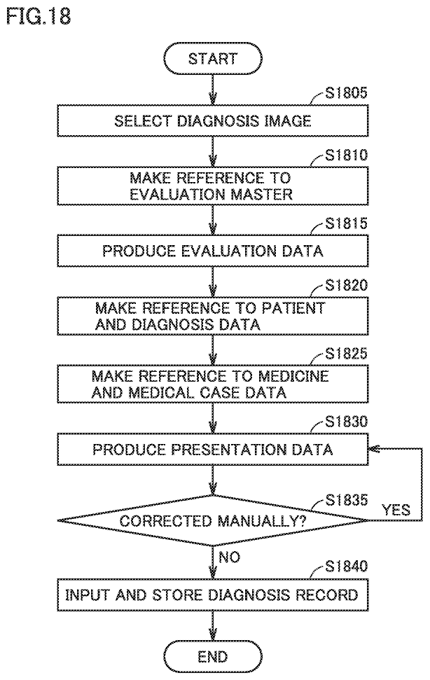

FIG. 18 shows exemplary processes up to the cornea diagnosis and the presentation with the combined image by ophthalmological diagnosis system 100 according to the present embodiment. The following describes a procedure in the processes for the diagnosis and the presentation with the combined image on the hardware of diagnosis device 102 with reference to FIG. 18. In a certain aspect, the various types of tables of FIG. 4 may be stored as data in secondary storage device 13 and may be referenced to by CPU 11. Further, in a certain aspect, the various types of functional units shown in FIG. 4 may be loaded as programs on primary storage device 12 and may be executed by CPU 11.

In a step S1805, CPU 11 serves as diagnosis evaluation unit 405 to select a combined image to be used for the diagnosis from the combined images for the diagnosis generated in the flow of FIG. 12. It should be noted that CPU 11 may select a plurality of combined images. When performing a diagnosis using the plurality of combined images, CPU 11 may use the average value of the diagnosis results of the respective combined images as the evaluation value, or may present examination results of the respective combined images on monitor 103 in the form of a slide.

In a step S1810, CPU 11 makes reference to evaluation master 410 on secondary storage device 13. Evaluation master 410 is a parameter for evaluating the size, depth, or the like of the injury on the surface of the cornea.

In a step S1815, CPU 11 serves as diagnosis evaluation unit 405 to evaluate the combined image selected in step S1805. When making the evaluation, CPU 11 uses the parameter read from evaluation master 410. The process of step S1815 corresponds to the process of FIG. 13 or 14.

In a step S1820, CPU 11 makes reference to patient master 411 and diagnosis data 413 on secondary storage device 13. CPU 11 uses the respective pieces of data read from patient master 411 and diagnosis data 413 to present the respective pieces of data as patient information 1702 and diagnosis history 1703 in FIG. 17.

In a step S1825, CPU 11 makes reference to medicine master 412 and medical case data 414 on secondary storage device 13. CPU 11 uses the respective pieces of data read from medicine master 412 and medical case data 414 to generate medicine administration information 1704, related data 1705, and proposal information 1706 in FIG. 17.

In a step S1830, CPU 11 serves as presentation information generation unit 407 to generate a diagnosis result screen. It should be noted that in the case of making the cornea diagnosis in the plurality of combined images, CPU 11 may present the diagnosis results for the plurality of combined images in cornea diagnosis image 1701. The manner of the presentation of the diagnosis results of the plurality of combined images is not limited to a specific manner For example, the diagnosis results may be presented in the form of a slide or a list.

In a step S1835, CPU 11 serves as editing UI 408 to receive a correction process from the user. When CPU 11 receives the correction process from the user (YES in step S1835), CPU 11 transitions the control to step S1830, and produces the presentation data again by reflecting the corrected content. When the correction process is not received from the user (NO in step S1835), CPU 11 transitions the control to step S1840. The process of step S1835 corresponds to the process of FIG. 15 or 16.

In a step S1840, CPU 11 serves as editing UI 408 to receive a diagnosis record input from the user. Then, CPU 11 adds the diagnosis record to diagnosis data 413.

By executing the processes of FIG. 18, CPU 11 can make a quantitative evaluation using various types of parameters in the cornea diagnosis using the combined image. Further, by presenting the various types of related information together with the cornea diagnosis result on monitor 103, CPU 11 supports an activity of the doctor or researcher.

<G. Reusability of Various Types of Information>

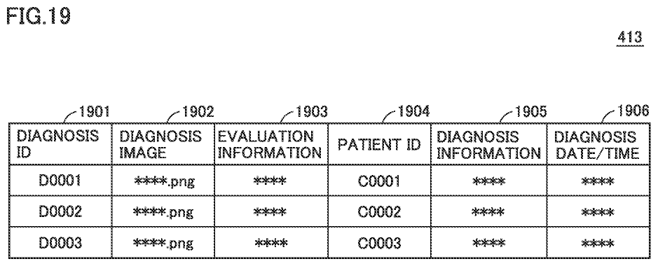

FIG. 19 is a diagram showing exemplary diagnosis data 413 according to the present embodiment. In order to improve reusability of the data, diagnosis data 413 separately stores various types of data to be presented on monitor 103 of FIG. 17. Referring to FIG. 19, diagnosis data 413 includes a diagnosis ID 1901, a diagnosis image 1902, evaluation information 1903, a patient ID 1904, diagnosis information 1905, and a diagnosis date/time 1906.

Diagnosis ID 1901 is an identifier for uniquely identifying an individual diagnosis. Diagnosis image 1902 is the combined image for the diagnosis generated in the process of FIG. 12 or is a path to the storage location of the combined image. It should be noted that the file format of the combined image is not limited, and may be, for example, a JPEG (Joint Photographic Experts Group) format, a PNG (Portable Network Graphics) format, or an original image format. Evaluation information 1903 is the data generated in FIG. 18, and is information to be superimposed on the combined image for the diagnosis for the sake of presentation.

Evaluation master 410 is improved in precision by receiving feedback of correction on evaluation information using editing UI 408. Therefore, by storing diagnosis image 1902 separately from evaluation information 1903 in diagnosis data 413, CPU 11 can examine the combined image for past diagnosis again using evaluation master 410 improved in precision.

Patient ID 1904 is used as a key for searching patient master 411 for information of the patient subjected to the diagnosis. Diagnosis information 1905 represents a content recorded by the doctor during the diagnosis. Diagnosis date/time 1906 represent a date/time when the diagnosis is made.

By analyzing evaluation information 1903 and diagnosis information 1905, CPU 11 can find a tendency of diagnosis results by diagnosis device 102 and a tendency of diagnosis results by the doctor, thereby facilitating proposal of treatment and presentation of related information in next and subsequent diagnoses.

As described above, diagnosis data 413 individually includes: the combined image for diagnosis (diagnosis image 1902) generated by diagnosis device 102; the evaluation information (evaluation information 1903) generated by diagnosis device 102; and the content (diagnosis information 1905) recorded by the doctor. Therefore, the reusability of the various types of data can be improved.

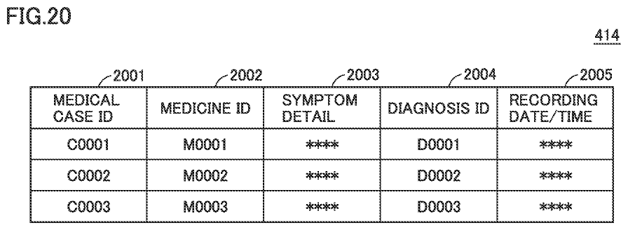

FIG. 20 is a diagram showing exemplary medical case data 414 according to the present embodiment. Medical case data 414 is a table in which information related to each medical case is recorded, and is mainly used for analysis as to what tendency is observed with respect to administration of a medicine. Referring to FIG. 20, medical case data 414 includes a medical case ID 2001, a medicine ID 2002, a symptom detail 2003, a diagnosis ID 2004, and a recording date/time 2005.

Medical case ID 2001 is an identifier for uniquely identifying an individual medical case. Medicine ID 2002 is an identifier for uniquely identifying a used medicine. CPU 11 uses medicine ID 2002 as a search key to perform search in medicine master 412.

The symptom detail represents a specific content of a symptom. It should be noted that different medical case IDs 2001 may be associated with the same symptom detail 2003. This means that the same symptom was observed multiple times.

Diagnosis ID 2004 is an identifier for uniquely identifying a diagnosis content associated with medical case ID 2001. CPU 11 uses diagnosis ID 2004 as a search key to perform search in diagnosis data 413. Recording date/time 2005 represent a date/time when the medical case was recorded. As described above, medical case data 414 stores a medicine, diagnosis information, and the like for each medical case, thereby facilitating an analysis on progression of treatment with regard to each medicine.

<H. Exemplary Applications>

Ophthalmological diagnosis system 100 according to the present embodiment is not limited to the implementation shown in FIG. 1. Various embodiments may be employed depending on applications, such as cooperation with machine learning or use at a plurality of locations. Hereinafter, exemplary applications of ophthalmological diagnosis system 100 will be described.

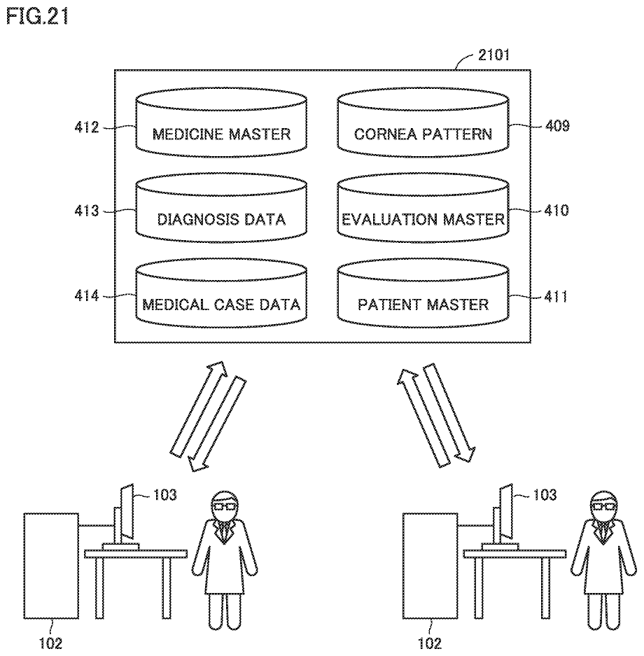

FIG. 21 is a diagram showing a first exemplary configuration of the ophthalmological diagnosis system according to the present embodiment. In the example shown in FIG. 21, a plurality of diagnosis devices 102 are connected to a server device 2101 via a network. Server device 2101 includes cornea pattern 409, evaluation master 410, patient master 411, medicine master 412, diagnosis data 413, and medical case data 414. Diagnosis device 102 communicates with server device 2101 to make reference to various types of table information during diagnosis recording. Further, diagnosis device 102 provide the diagnosis data with a diagnosis record including metadata such as patient information and information of a doctor having made the diagnosis, and transmits it to server device 2101. Server device 2101 stores the received diagnosis record into the various types of tables based on the metadata.

It should be noted that in the example shown in FIG. 21, each diagnosis device 102 does not need to include cornea pattern 409, evaluation master 410, patient master 411, medicine master 412, diagnosis data 413, and medical case data 414. Further, server device 2101 may provide an interface as a web application. In this case, diagnosis device 102 can access a function or data provided by server device 2101 using a browser without installing dedicated software.

The exemplary configuration of FIG. 21 allows a plurality of doctors in a hospital to use a common diagnosis system, thereby facilitating maintenance of consistency of diagnosis record and reduction of device introduction cost. Further, when the function of server device 2101 is provided as a web application, the system administrator only needs to perform maintenance update of server device 2101, thus also attaining reduction of running cost.

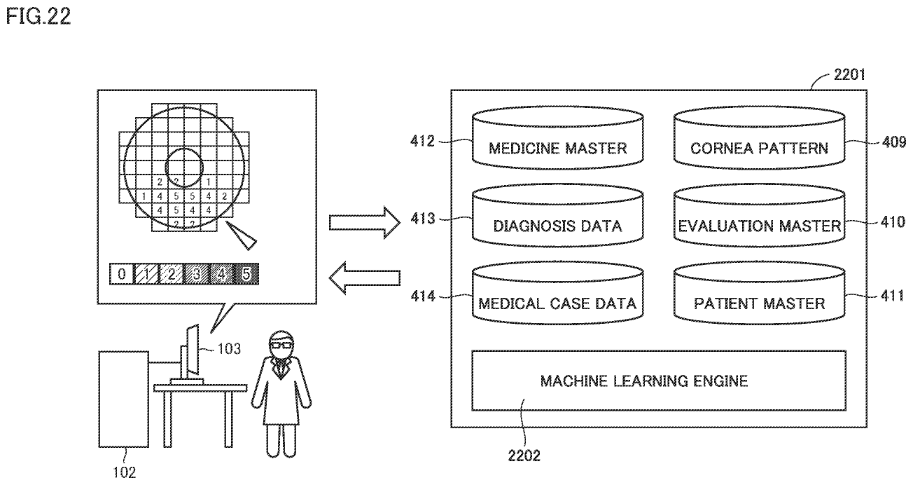

FIG. 22 is a diagram showing a second exemplary configuration of the ophthalmological diagnosis system according to the present embodiment. In the example shown in FIG. 22, one or more diagnosis devices 102 are connected to a server device 2201 via a network. Server device 2201 includes cornea pattern 409, evaluation master 410, patient master 411, medicine master 412, diagnosis data 413, medical case data 414, and a machine learning engine 2202. Diagnosis device 102 communicates with server device 2201 to make reference to various types of table information during diagnosis recording. Further, diagnosis device 102 provides the diagnosis data with a diagnosis record including metadata such as patient information and information of the doctor having made the diagnosis, and transmits it to server device 2201. Server device 2201 stores the received diagnosis record into the various types of tables based on the metadata.

Machine learning engine 2202 can update evaluation master 410 based on a history of correction operation using editing UI 408, thereby improving precision in the evaluation on the combined image. Similarly, machine learning engine 2202 can update cornea pattern 409 based on the history of correction operation using editing UI 408 and the still image generated during the diagnosis, thereby improving precision in the cornea detection. Further, machine learning engine 2202 may process diagnosis data 413 and medical case data 414 as training data so as to update related data 1705 and proposal information 1706 to be presented on monitor 103.

Further, as with the example of FIG. 21, server device 2201 may provide an interface as a web application. In this case, diagnosis device 102 can access a function and data provided by server device 2201 using a browser without installing dedicated software. Further, diagnosis device 102 and server device 2201 may be integrated.

In the exemplary configuration of FIG. 22, the diagnosis information input by the user and the history of correction operation can be used as training data for machine learning engine 2202. Hence, precision in diagnosis can be continuously improved.

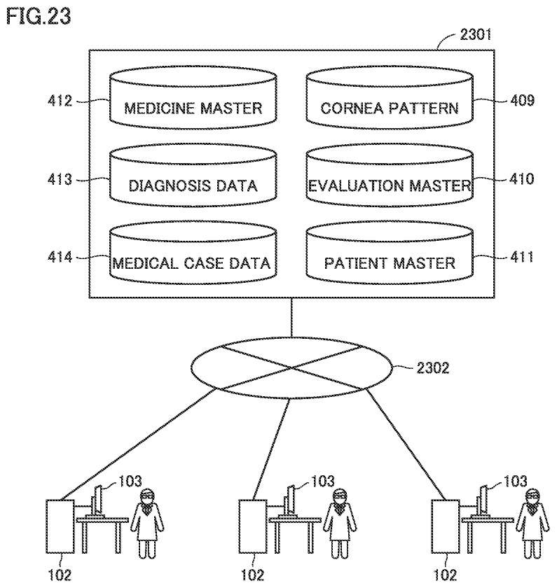

FIG. 23 is a diagram showing a third exemplary configuration of the ophthalmological diagnosis system according to the present embodiment. In the example shown in FIG. 23, a plurality of diagnosis devices 102 are connected to a server device 2301 via a public network 2302.

As with the example of FIG. 21, server device 2301 includes cornea pattern 409, evaluation master 410, patient master 411, medicine master 412, diagnosis data 413, and medical case data 414. Diagnosis device 102 communicates with server device 2301 to make reference to various types of table information during diagnosis recording. Further, diagnosis device 102 provides the diagnosis data with metadata such as patient information and information of the doctor having made diagnosis, and transmits it to server device 2301. Server device 2301 updates the various types of tables based on the received diagnosis data and the metadata.

Further, as with the other exemplary configurations described above, server device 2301 may provide an interface as a web application. In this case, diagnosis device 102 can access a function and data provided by server device 2201 using a browser without installing dedicated software. Further, diagnosis device 102 and server device 2301 may be integrated.

In the exemplary configuration of FIG. 23, results of clinical trials in a hospital and the like can be immediately fed back to a pharmaceutical company. Further, the doctor can make reference to latest medicine information of the pharmaceutical company.

The embodiments disclosed herein are illustrative and non-restrictive in any respect. The scope of the present invention is defined by the terms of the claims, rather than the embodiments described above, and is intended to include any modifications within the scope and meaning equivalent to the terms of the claims.

### REFERENCE SIGNS LIST

11: CPU; 12: primary storage device; 13: secondary storage device; 14: external device interface; 15: input interface; 16: output interface; 17: communication interface; 100: ophthalmological diagnosis system; 101: camera; 102: diagnosis device; 103: monitor; 205, 1905: diagnosis information; 206: related information; 401: moving image processing unit; 402: moving image analysis unit; 403: image analysis unit; 404: combining processing unit; 405: diagnosis evaluation unit; 406: communication processing unit; 407: presentation information generation unit; 408: editing UI; 409: cornea pattern; 410: evaluation master; 411: patient master; 412: medicine master; 413: diagnosis data; 414: medical case data; 702, 802, 902, 1002: combined image; 1501: diagnosis result; 1502, 1602: cursor; 1503: evaluation value selector; 1601: image; 1701, 1902: diagnosis image; 1702: patient information; 1703: diagnosis history; 1704: medicine administration information; 1705: related data; 1706: proposal information; 1707: comprehensive evaluation; 1901, 2004: diagnosis ID; 1903: evaluation information; 1904: patient ID; 1906: diagnosis date/time; 2001: medical case ID; 2002: medicine ID; 2003: detail; 2005: recording date/time; 2101, 2201, 2301: server device; 2202: machine learning engine; 2302: public network.

1. An ophthalmological diagnosis device for providing information to assist in making a diagnosis on a cornea, the ophthalmological diagnosis device comprising:
an input unit that receives an input of video image data from an external device;
an output unit that outputs image data;
a storage unit that stores an evaluation criterion for the injury of the cornea; and
a processing unit that processes data, wherein
the processing unit generates a diagnosis image based on a position of the cornea in each still image of the video image data,
the processing unit divides the cornea captured in the generated diagnosis image into a plurality of regions, and evaluates an injury in each of the divided regions based on the evaluation criterion,
the processing unit generates diagnosis information in which the diagnosis image and information about the evaluation on the injury in each of the regions are superimposed on each other, and
the processing unit outputs the diagnosis information through the output unit.

2. The ophthalmological diagnosis device according to claim 1, wherein
the generating of the diagnosis image includes
extracting a reference still image from the video image data based on the position of the cornea, and
combining the reference still image with another still image extracted from the video image data, by superimposing the other still image on the reference still image based on positions of an iris and the injury of the cornea captured in the reference still image.

3. The ophthalmological diagnosis device according to claim 2, wherein
the generating of the diagnosis image further includes
comparing portions of the reference still image and the superimposed still image, the portions of the reference still image and the superimposed still image being estimated to represent the same position on the cornea, and
removing, from the diagnosis image, a portion determined to have a dust based on a contrast ratio or high-frequency component in each of the portions of the still images estimated to represent the same position.

4. The ophthalmological diagnosis device according to claim 2, wherein the generating of the diagnosis image further includes a process of using, for alignment of the reference still image and the superimposed still image, a wrinkle of a conjunctiva captured in each of the reference still image and the superimposed still image.

5. The ophthalmological diagnosis device according to claim 2, wherein the generating of the diagnosis image further includes a process of determining portions of the reference still image and the superimposed still image as light reflected by a conjunctiva, and avoiding the portions determined as the light reflected by the conjunctiva from being included in the diagnosis image, the portions determined as the light reflected by the conjunctiva being portions in each of which a luminance on the conjunctiva is more than a predetermined value.

6. The ophthalmological diagnosis device according to claim 2, wherein the generating of the diagnosis image further includes a process of emphasizing a portion of the captured cornea having a high contrast ratio or high-frequency component in the diagnosis image after the combining.

7. The ophthalmological diagnosis device according to claim 1, wherein
the generating of the diagnosis information further includes
detecting position and size of the cornea captured in the diagnosis image,
dividing the cornea into the plurality of regions in a form of a grid based on the detected position and size of the cornea, and
evaluating the injury in each of the regions divided in the form of the grid, based on a contrast ratio or high-frequency component in the region.

8. The ophthalmological diagnosis device according to claim 7, wherein the generating of the diagnosis information further includes a process of superimposing a score on each of the regions divided in the form of the grid in the diagnosis image, the score being based on the evaluation on the injury in the region.

9. The ophthalmological diagnosis device according to claim 7, wherein the generating of the diagnosis information further includes a process of superimposing a frame line on each of the regions divided in the form of the grid in the diagnosis image, the frame line having a color that is based on the evaluation on the injury in the region.

10. The ophthalmological diagnosis device according to claim 7, wherein the generating of the diagnosis information further includes a process of calculating a comprehensive score for the evaluations on the injury in the regions.

11. The ophthalmological diagnosis device according to claim 7, wherein the generating of the diagnosis information further includes a process of blurring an iris portion captured in the diagnosis image.

12. The ophthalmological diagnosis device according to claim 1, further comprising a communication unit that communicates through a network, wherein the processing unit transmits, to an external server via the communication unit, the diagnosis information to which metadata is added.

13. The ophthalmological diagnosis device according to claim 1, wherein
the storage unit further stores data about medicine administration,
the processing unit obtains the data about the medicine administration from the storage unit, and
the processing unit generates information of a medicine related to the diagnosis information, based on the diagnosis information and the data about the medicine administration.
